(19) United States
(12) Patent Application Publication
(10) Pub. No.: US 2021/0125708 A1
(43) Pub. Date: Apr. 29, 2021

(54) PATIENT SCHEDULING SYSTEM

(71) Applicant: Simon Patrick Ivor Treissman, Kamloops (CA)

(72) Inventor: Simon Patrick Ivor Treissman, Kamloops (CA)

(21) Appl. No.: 16/663,093

(22) Filed: Oct. 24, 2019

(51) Int. Cl. G16H 40/20; G16H 80/00; G16H 40/67

(52) U.S. Cl. CPC G16H 40/20; G16H 40/67; G16H 80/00

(57) ABSTRACT

A method for scheduling surgical procedures comprises storing information pertaining to a plurality of surgical procedures in a database, the information including required personnel and time of the surgical procedure, providing at least one access to authorized users over a network wherein the user inputs at staid at least one access a new surgical procedure to be performed and receiving from the at least one access inputted information concerning the new surgical procedure. The method further comprises automatically reorganizing, utilizing a processor, the plurality of surgical procedures to include the new surgical procedure according to at least one algorithm, automatically generating a message containing information on at least one of the plurality of surgical procedures and transmitting the message to all required personnel over the network in real time.

### BACKGROUND OF THE INVENTION

### 1. Field of Invention

The present invention relates generally to health care management and in particular to a system and method for scheduling patient procedures.

### 2. Description of Related Art

Emergency operating room scheduling is a difficult and complex process whereby staff, operating rooms and other equipment is scheduled according to the urgency of the procedure and other considerations. Current hospital scheduling systems primarily rely on pen and paper and whiteboards for scheduling surgeries and other procedures. Such systems are time consuming requiring personnel dedicated to balancing the competing interests of doctors, patients and hospital resources. Such time constraints required to manage and operate such a system are unacceptably slow and prone to missed information due to miscommunication or time delays.

### SUMMARY OF THE INVENTION

According to a first embodiment of the present invention there is disclosed a method for scheduling surgical procedures comprising storing information pertaining to a plurality of surgical procedures in a database, the information including required personnel and time of the surgical procedure, providing at least one access to authorized users over a network wherein the user inputs at staid at least one access a new surgical procedure to be performed and receiving from the at least one access inputted information concerning the new surgical procedure. The method further comprises automatically reorganizing, utilizing a processor, the plurality of surgical procedures to include the new surgical procedure according to at least one algorithm, automatically generating a message containing information on at least one of the plurality of surgical procedures and transmitting the message to all required personnel over the network in real time.

The message contains a request for confirmation of attendance of the required personnel at the at least one of the plurality of surgical procedures wherein the method further comprises receiving a reply from each of the required personnel over the network indicating one of attendance or rejection. When all required personnel have indicated a positive attendance, the procedure is confirmed for the indicated location and time. The method may further comprise providing an output to an operator if a reply to the message is not received for further follow by the operator.

The inputted information concerning new surgical procedure includes a surgical procedure type and a target wait time. The target wait time is measured from a time when the inputted information concerning new surgical procedure is inputted into the at least one access. The inputted information concerning new surgical procedure includes an indication of urgency. The indication of urgency for that surgical procedure is from one of a plurality of classifications.

The automatically reorganizing comprises ordering the plurality of surgical procedures according to priority based on the target wait time and surgical procedure type.

According to a further embodiment of the present invention there is disclosed a system for scheduling surgical procedures comprising a database adapted to store information pertaining to a plurality of surgical procedures in a database, the information including required personnel and time of the surgical procedure and at least one access to authorized users over a network wherein the user inputs at staid at least one access a new surgical procedure to be performed. The system further comprises a processor adapted to receive from the at least one access, inputted information concerning the new surgical procedure wherein the processor is adapted to automatically reorganize the plurality of surgical procedures to include the new surgical procedure according to at least one algorithm wherein the processor is further adapted to automatically generating a message containing information on at least one of the plurality of surgical procedures and a transmitter operably connected to the processor operable to transmit the message to all required personnel over the network in real time.

The system may further comprise a receiver operable to receive a reply from the required personnel over the network indicating one of attendance or rejection. The message is transmitted via short messaging service.

The system may further comprise an operator interface operable to display to an operator if no reply is received from the required personnel. The operator interface is operable to receive a modification to an order of the plurality of surgical procedures.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view,

FIG. 1 is an illustration of a system for scheduling surgical procedures according to a first embodiment of the present invention.

FIG. 2 is a block diagram of the system of FIG. 1.

FIG. 3 is a block diagram of a method for receiving and ordering inputted surgical procedures into the system of FIG. 1 FIG. 4 is a block diagram of a method for contacting a scheduling surgical personnel for use in the system of FIG. 1.

FIG. 5 is an illustration of a data structure for use in the system of FIG. 1.

FIG. 6 is an illustration an input screen of the system of FIG. 1.

FIG. 7 is an illustration of a case management screen for use in the system of FIG. 1.

FIG. 8 is an illustration of a case management screen for use in the system of FIG. 1.

### DETAILED DESCRIPTION

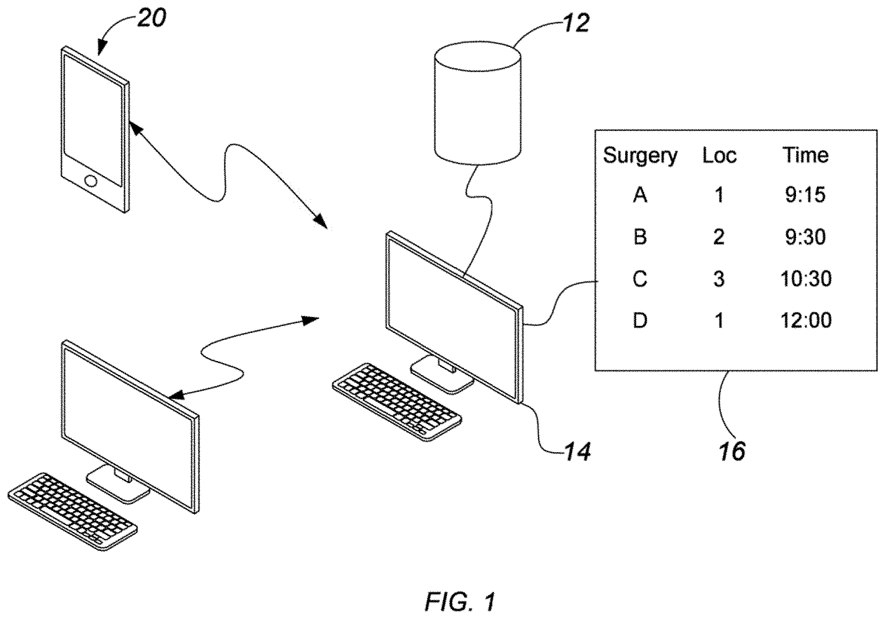

Referring to FIG. 1, a system for scheduling surgical procedures according to a first embodiment of the invention is shown generally at 10. The system comprises a database 12 in communication with a processor 14 which is operable to receive a plurality of surgical procedures inputted into a network access device (generally indicated at 20). The processor then sorts and organizes the surgical procedures by priority according to an algorithm to create a schedule 16 of such procedures which will be set out below and sends messages to the required personnel to confirm their attendance at that procedure.

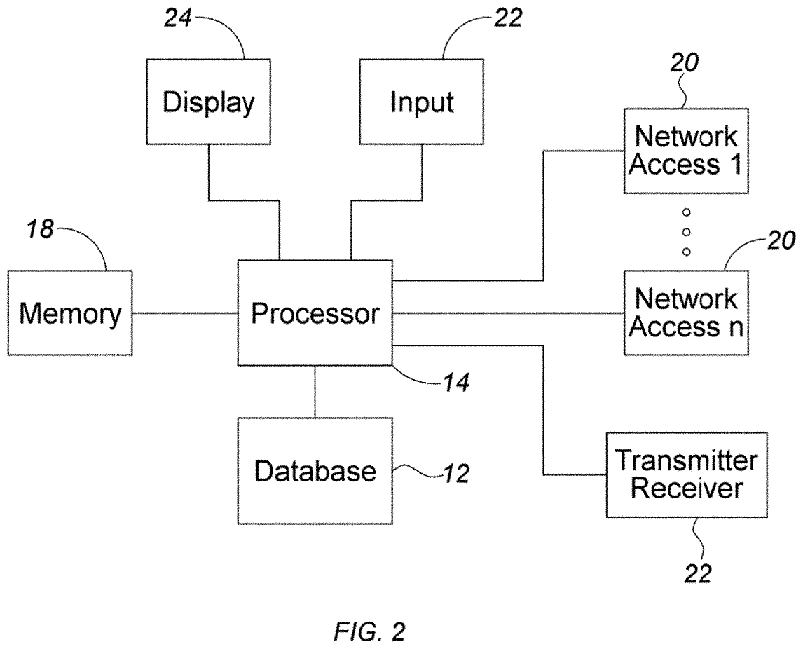

Turning now to FIG. 2, the system 10 comprises the processor 14, and memory 18 that stores machine instructions that, when executed by the processor 14, cause the processor 14 to perform one or more of the operations and methods described herein. The processor 14 may optionally contain a cache memory unit for temporary local storage of instructions, data, or computer addresses. The system 10 further includes a database 12 or data storage of any conventional type operable to store information such as a plurality of light control profiles relating to a variety of plant or photosynthetic organisms to be targeted and may optionally include an input transmitter/receiver 22 and display 24 for receiving and displaying inputs from an administrator or user. As outlined above, the processor 14 is adapted to organize and schedule a plurality of surgical procedures according to priority.

More generally, in this specification, the term “processor” is intended to broadly encompass any type of device or combination of devices capable of performing the functions described herein, including (without limitation) other types of microprocessors, microcontrollers, other integrated circuits, other types of circuits or combinations of circuits, logic gates or gate arrays, or programmable devices of any sort, for example, either alone or in combination with other such devices located at the same location or remotely from each other. Additional types of processor(s) will be apparent to those ordinarily skilled in the art upon review of this specification, and substitution of any such other types of processor(s) is considered not to depart from the scope of the present invention as defined herein. In various embodiments, the processor 14 can be implemented as a single-chip, multiple chips and/or other electrical components including one or more integrated circuits and printed circuit boards.

Computer code comprising instructions for the processor(s) to carry out the various embodiments, aspects, features, etc. of the present disclosure may reside in the memory 18. The code may be broken into separate routines, products, etc. to carry forth specific steps disclosed herein. In various embodiments, the processor 14 can be implemented as a single-chip, multiple chips and/or other electrical components including one or more integrated circuits and printed circuit boards. The processor 14 together with a suitable operating system may operate to execute instructions in the form of computer code and produce and use data. By way of example and not by way of limitation, the operating system may be Windows-based, Mac-based, or Unix or Linux-based, among other suitable operating systems. Operating systems are generally well known and will not be described in further detail here.

Memory 18 may include various tangible, non-transitory computer-readable media including Read-Only Memory (ROM) and/or Random-Access Memory (RAM). As is well known in the art, ROM acts to transfer data and instructions uni-directionally to the processor 14, and RAM is used typically to transfer data and instructions in a bi-directional manner. In the various embodiments disclosed herein, RAM includes computer program instructions that when executed by the processor 14 cause the processor 14 to execute the program instructions described in greater detail below. More generally, the term “memory” as used herein encompasses one or more storage mediums and generally provides a place to store computer code (e.g., software and/or firmware) and data. It may comprise, for example, electronic, optical, magnetic, or any other storage or transmission device capable of providing the processor 14 with program instructions. Memory 18 may further include a floppy disk, CD-ROM, DVD, magnetic disk, memory chip, ASIC, FPGA, EEPROM, EPROM, flash memory, optical media, or any other suitable memory from which processor 14 can read instructions in computer programming languages.

It will be appreciated that the network access device 20 may be any commonly known user device, such as, by way of non-limiting example, a tablet, laptop computer, smartphone, PDA, ultra mobile PC (UMPC), desktop computer, server. etc. It will be understood that the architecture herein is provided for example purposes only and does not limit the scope of the various implementations of the communication systems and methods. As will be further described below, the network access device 20 is adapted access the processor through the network either a through a program installed thereon or operated through a web application as are commonly known. The network access device 20 may communicate with the processor 14 via a network as are commonly known including local area networks (LAN), wide area networks (WAN), internet and cellular networks and radio transmissions. The system 10 includes a transmitter/receiver 22 for transmitting a message to one or more of the users. In particular, the transmitter/receiver may comprise any known transmitter/receiver including for transmission via text, email, short message service (SMS), multimedia messaging service (MMS) or the like.

The instructions to the processor 14 may comprise one or more algorithm that determines how the surgical procedures are to be ordered depending upon several factors. In particular, the processor 14 organizes the surgery wait list by inter collating the various wait time targets into an appropriate order as they each countdown. In particular, the processor organizes the surgical procedures based upon the time urgency of the procedure as based upon the surgery type, and the target wait time as entered by the surgeon or other user entering the data. In particular, such inter collating comprises receiving the desired wait time entered for each surgery and then determining a target time for that surgery to take place. The processor 14 orders the procedures in terms of their target time where it will be appreciated that the clock for previously entered surgeries is currently running. Therefore the actual location of each surgery in priority will be a combination of the desired wait time as well as the time at which the surgery is entered in so much as they both impact the actual target time for that surgery.

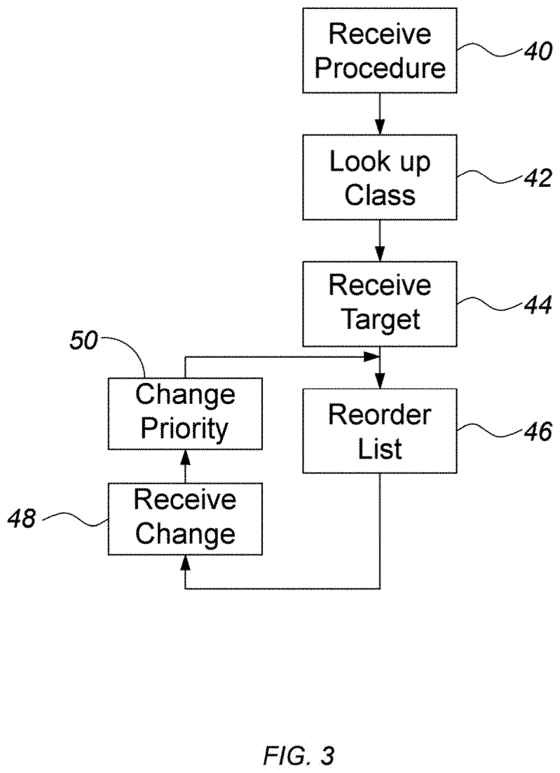

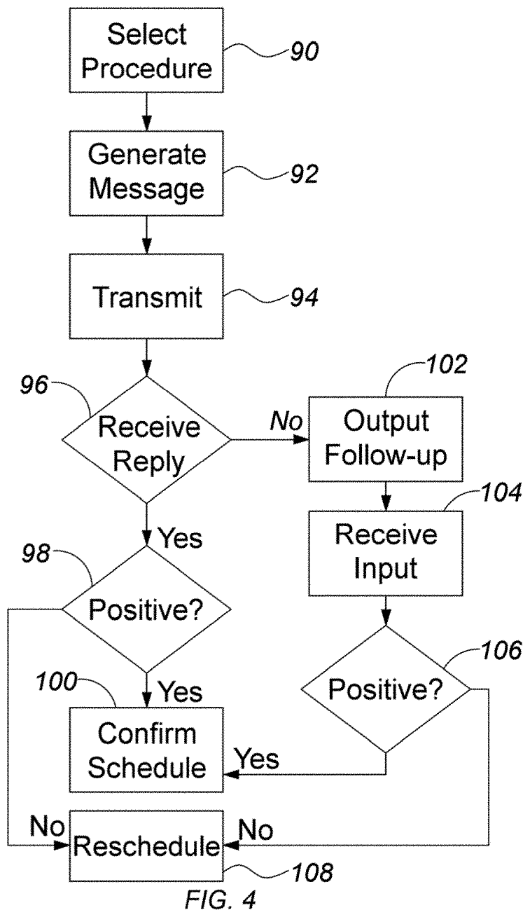

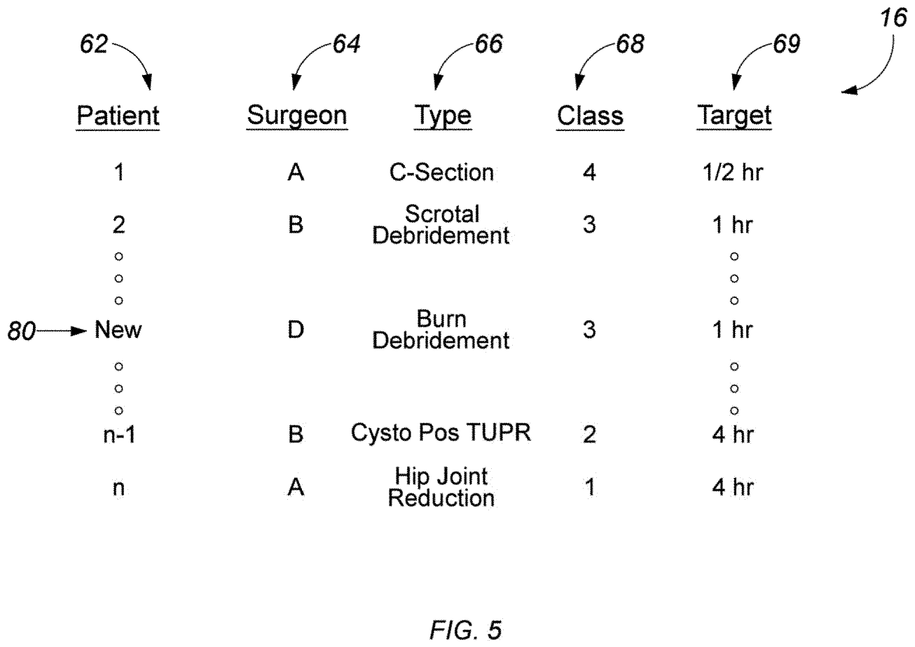

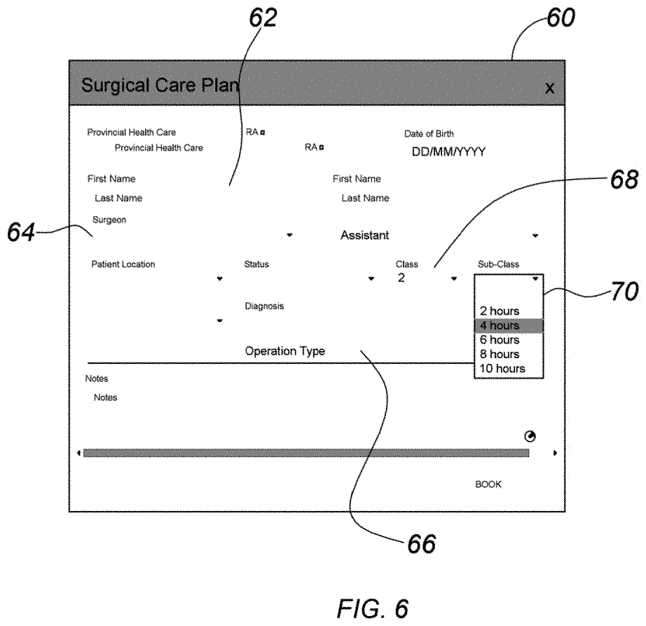

Turning now to FIGS. 3 and 6, a flow chart and data input screen of the procedure input process for the system 10 is illustrated. At step 40, the system receives an input of a procedure as inputted into the inputs screen 60. The input screen may be displayed on a network access device 20. In particular, the input screen 60 includes data entry fields for the patent name 62 and other particulars, the surgeon identification 64 and the operation type 66. The operation type 66 may comprise a list that is stored within the database 12 which has a standard priority class 68 and one or more optional target wait times 69. Optionally, a surgeon may be able to enter their own descriptions and categorize the priority class and target wait times. As illustrated in FIG. 3, the processor 14 may system look up the class entry 68 from the database 12 from the surgery at step 42 and then receives a suggested target wait time 69 which can be selected by the surgeon in step 44. The processor 14 then utilizes the class and sub-class information to determine the position of that procedure within the overall list in step 46 as illustrated in FIG. 5 where a new procedure 80 between a plurality of existing procedures.

As illustrated in FIG. 3, an operator or administrator may re-order one or more of the procedures at their discretion as illustrated in FIG. 48. It will be appreciated that such re-ordering will be reserved for personnel having higher levels of decision making power and that such decisions will be based upon factors which will be based on their experience and discretion. In such cases, the processor 14 may utilize such re-ordering to adjust the priority level of that procedure type for future uses in accordance with the algorithm.

Turning now to FIG. 4, the processor 14 is adapted to transmit a message to each required personnel for that particular procedure. In particular, it will be appreciated that such required personnel may include surgeons, nurses, technicians, anaesthesiologists or any other required persons for that particular procedure. As illustrated in step 90, the processor selects one or more case as the scheduled time for that procedure is approaching as determined by the schedule 16. In particular, the processor may select such cases at a predetermined time before their intended start or may optionally select one or more procedures in a batch at a set time interval. The processor 14 then generates a message in step 92 to advise the personnel of the time and location of the upcoming surgery which is transmitted through the transmitter/receiver 22 in step 94. The message is also selected to include a required response accepting or rejecting the scheduled procedure. If no response is received at the transmitter/receiver 22, the processor 14 may be adapted to provide a prompt to an operator or administrator to contact that personnel in step 102 or optionally to send another message. In step 104, the system may be adapted to enable the operator or administrator to manually enter a response for that personnel into the system which is positive or negative as assessed in step 106.

When a response is received, it is analysed in step 98 by the processor. It will be appreciated that the response may include any positive or negative response such as a yes or no, confirm/reject by way of non-limiting example. In step 96, the processor 14 determines if a positive or negative response was received at the transmitter/receiver 22 from each required personnel. If a positive response was received, either through step 98 or inputted by an administrator in step 106, the procedure is confirmed in the schedule in step 100. By way of non limited example, when a user provides an affirmative or positive response the system may display a message on the screen of an administrator or other user that “Dr. (specified) is on their way to the operating room”. If the response is negative, either as responded to by the user or inputted by the administrator or a response is not received, a further prompt is provided to the administrator to contact that Dr. to discuss seek further input on whether the surgery must be rescheduled or other arrangements and staffing provided.

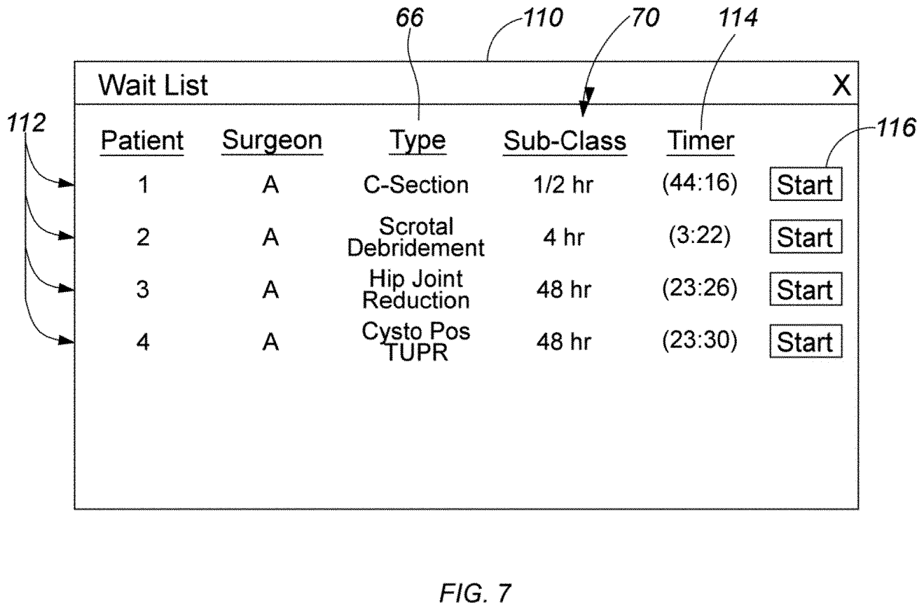

Turning now to FIG. 7, a status screen 110 which may be presented to a particular user is illustrated. As illustrated in FIG. 7, a summary of upcoming surgeries is shown for only a single surgeon however, more than one surgeon may also be summarized for analytical purposes. As illustrated in FIG. 7, upcoming surgery is illustrated 112 and ordered in descending order of precedence. In particular, the status screen 110 indicates the type of surgery 66, the class and/or subclass, 68 or 70 and the timer 114 indicating how long it is to the end of the target wait time. As illustrated in FIG. 7, a surgery which is overdue or late may also be indicated either with a negative figure or in a different colour. The status screen 110 may also include a start button for each surgery to indicate when the case has been called to the OR which may be selected by a user to indicate that the case has been called to the operating room. Once the start button has been pressed by a user, the processor 14 will store the start time and any remaining wait time (or overdue time) remaining for future use.

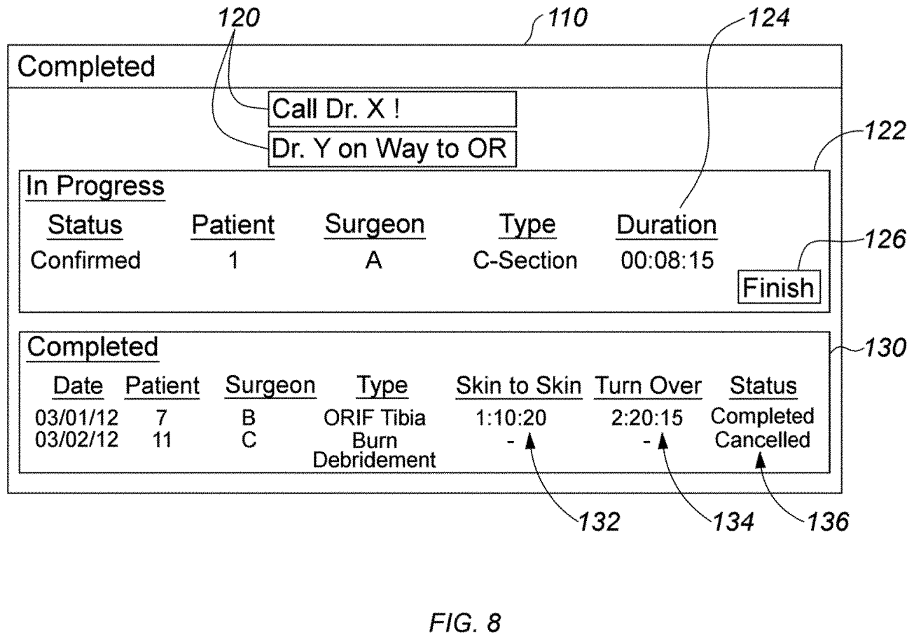

Turning now to FIG. 8, a screen shot 10 of the system as observed by a user such as an administrator or clerk is illustrated in which the completed and in-progress surgeries are illustrated. As illustrated in FIG. 8, one or more messages or alerts 120 may be displayed indicating to the user if a particular surgeon has replied to their paging message as set out above. It will be appreciated that such alerts may indicate to the user, if the surgeon has not replied to their message, if they have replied with a positive message or if they have replied with a negative response. The alert may also prompt the user to take further steps such as indicating that they contact the surgeon for further information or contact one or other personnel.

The surgeries in-progress may be displayed in an in-progress window 122 wherein the information as set out above continues to be displayed along with a timer indicating the current duration of the surgery 124 and a finish button 126 for a surgeon, administrator or clerk to indicate that the surgery has completed. A surgeon or other user may also be prompted to input a skin to skin time in an input popup or other window (not shown) corresponding to the time of the surgery from the initial cut to finishing the final stitches. The processor 14 utilizes the start time of the surgery, finish time of the surgery and the inputted skin-to-skin time to calculate both an intercase time (being the duration time from when the start button is pushed to when the finish button is pushed) as well as the turnover time (being the intercase time minus the entered skin-to-skin time). It will be appreciated that the processor 14 may store intercase, skin-to-skin and turnover times for each procedure to be reviewed on an individual, department and hospital wide basis by hospital administrators or the like.

As further illustrated in FIG. 8, the user screen may also display competed surgeries 130. The completed surgeries screen may include the information for each procedure as set out above and may also display the skin-to-skin and turnover times, 132 and 134, respectively for each surgery. The completed screen may also indicate if the surgery was completed, cancelled or any other indication of its status 136. If the surgery is cancelled, a user may have the option of returning the surgery to the waitlist wherein the processor 14 will retrieve the time the procedure was originally entered as well as the desired wait time to calculate the original target time. This procedure may then be returned to the wait list in the proper order as it would have been had it not been called to the operating room by pressing the start button.

It will be appreciated that the database may require user authentication for each unique user as is commonly known. In particular, the options including viewing, creating or editing procedures may be limited or controlled via such user authentication.

The present system may permit analytics be performed from time to time or on an ongoing basis. In particular, as each procedure is entered and tracked through the system, an administrator may utilize such recorded data to analyse performance of the entire hospital or individual members and departments. Each surgeon or procedure type may be assessed to determine progress on improving efficiency or wait list times or to determine if any particular personnel are attempting to skew or artificially reduce their wait times by entering shortened time limits.

While specific embodiments of the invention have been described and illustrated, such embodiments should be considered illustrative of the invention only and not as limiting the invention as construed in accordance with the accompanying claims.

What is claimed is:

1. A method for scheduling surgical procedures comprising:
storing information pertaining to a plurality of surgical procedures in a database, said information including required personnel and time of the surgical procedure;
providing at least one access to authorized users over a network wherein said user inputs at staid at least one access a new surgical procedure to be performed;
receiving from said at least one access inputted information concerning said new surgical procedure;
automatically reorganizing, utilizing a processor, said plurality of surgical procedures to include said new surgical procedure according to at least one algorithm;
automatically generating a message containing information on at least one of said plurality of surgical procedures; and
transmitting said message to all required personnel over said network in real time.

2. The method of claim 2 wherein said message contains a request for confirmation of attendance of said required personnel at said at least one of said plurality of surgical procedures wherein said method further comprises receiving a reply from each of said required personnel over said network indicating one of attendance or rejection.

3. The method of claim 3 wherein said when all required personnel have indicated a positive attendance, said procedure is confirmed for the indicated location and time.

4. The method of claim 1 further comprising providing an output to an operator if a reply to said message is not received for further follow by said operator.

5. The method of claim 1 wherein said inputted information concerning new surgical procedure includes a surgical procedure type and a target wait time.

6. The method of claim 5 wherein said target wait time is measured from a time when said inputted information concerning new surgical procedure is inputted into said at least one access.

7. The method of claim 5 wherein said inputted information concerning new surgical procedure includes an indication of urgency.

8. The method of claim 7 wherein said indication of urgency for that surgical procedure is from one of a plurality of classifications.

9. The method of claim 5 wherein said automatically reorganizing comprises ordering said plurality of surgical procedures according to priority based on said target wait time and surgical procedure type.

10. A system for scheduling surgical procedures comprising:
a database adapted to store information pertaining to a plurality of surgical procedures in a database, said information including required personnel and time of the surgical procedure;
at least one access to authorized users over a network wherein said user inputs at staid at least one access a new surgical procedure to be performed;
a processor adapted to receive from said at least one access, inputted information concerning said new surgical procedure;
said processor adapted to automatically reorganize said plurality of surgical procedures to include said new surgical procedure according to at least one algorithm;
said processor further adapted to automatically generating a message containing information on at least one of said plurality of surgical procedures; and
a transmitter operably connected to said processor operable to transmit said message to all required personnel over said network in real time.

11. The system of claim 10 further comprising a receiver operable to receive a reply from said required personnel over said network indicating one of attendance or rejection.

12. The system of claim 11 wherein said message is transmitted via short messaging service.

13. The system of claim 10 further comprising an operator interface operable to display to an operator if no reply is received from said required personnel.

14. The system of claim 13 wherein said operator interface is operable to receive a modification to an order of said plurality of surgical procedures.
